(19) United States
(12) Patent Application Publication
(10) Pub. No.: US 2010/0323429 A1
(43) Pub. Date: Dec. 23, 2010

(54) Methods for purifying baculovirus

(76) Inventors: Yu-Chen Hu, Hsinchu City (TW); Guan-Yu Chen, Yonghe City (TW)

Correspondence Address: Andrews & Kurth, L.L.P., Suite 1100, 1350 I Street N.W., Washington, DC, 20005, US

(21) Appl. No.: 12/081,065

(22) Filed: Apr. 10, 2008

(51) Int. Cl. C12N 7/02

(52) U.S. Cl. 435/239

(57) ABSTRACT

The present invention provides a method for purifying baculovirus comprising: providing a baculovirus mixture containing a baculovirus and a liquid portion; replacing the liquid portion with a binding buffer by an ultrafiltration system to form a virus buffer; and purifying the baculovirus from the virus buffer using glycoprotein specific affinity chromatography. Therefore, use of the method of the present invention in the purification of baculovirus resulted in an enhanced discovery yield and improved purity of virus.

### BACKGROUND OF THE INVENTION

1. Field of the Invention

The invention relates to a method for purifying viruses, and more particularly to a method for purifying baculovirus with improved virus recovery yield and purity.

2. Description of the Prior Arts

Baculovirus has various hosts consisting of crustacean animals and insects, and can infect over 600 species of insects, mainly Lepidoptera. Baculovirus can be divided into 3 subgroups. Nuclear polyhedrosis viruses (NPV), one of these subgroup, can be cultured in stable cell lines with its unique infectious cycle and gene expression, suggesting the potential of NPV for being vectors for gene expression (Luckow, V. A. and Summers, M. D., (1988), Virology, 167(1): 56-71). The genetic material of NPV is double-stranded DNA with a size range between 80 to 200 kb and encapsulated by rod-shaped nucleocapsid. The genetic material encapsulated by the nucleocapsid is further covered by a lipoprotein envelope to form a baculovirus particle with a diameter of 40 to 50 nm and length of 200 to 400 nm. Bombyx mori nucleopolyhedrovirus (BmNPV) and Autographa californica nucleopolyhedrovirus (AcMNPV) belonging to genus NPV are currently the most commonly used baculovirus expression vector system.

Baculovirus expression vector system was established in 1983. After being developed for 2 decades, baculovirus expression vector systems have become a convenient expression system for producing exogenous recombinant protein (Luckow, V. A. and Summers, M. D., (1988), supra; Possee, R. D., (1997), Curr. Opin. Biotechnol., 8(5):569-72).

The most prominent advantage of baculovirus expression vector system is that insect cells infected by the recombinant baculovirus are eukaryotic cells. Since the insect cells possess a certain degree of post-translational modification, the recombinant proteins expressed by the expression system with insect cells resemble those expressed in mammalian cells with respect to their post-translational modification. The recombinant eukaryotic proteins produced by the baculovirus expression vector system have similar antigenicity, immunity and bioactivity with native proteins. Therefore, more and more researchers use this expression system to produce vaccines or recombinant antigens.

Baculovirus DNA cannot efficiently enter nuclei during a process of transducing into cells. To increase transducing efficiency in mammalian cells, viral tropism is altered by modifying, replacing or adding components to a virus surface to enhance the transducing efficiency. These components are often derived from other viruses having specific tropism. The most widely used component is G glycoprotein of the vesicular stomatitis virus (VSVG) (Ugai, H. et al., (2005), Biochem. Biophys. Res. Commun., 331(4):1053-60). It is reported that the expression of VSVG on baculovirus can enhance the transducing efficiency of the virus in mammalian cells.

For transducing viruses carrying a recombinant gene into host cells, purification of viruses is required before usage. Impurities contained in solution surrounding the virus cause a reduction in transduction efficiency of the virus or negatively affect bioorganism safety. To remove the impurities, the purification of viruses includes processes of concentration, exchange of solution surrounding the virus and purification. The efficiency of each process can be evaluated by recovery yield and purity of virus before and after each process. In addition, time and convenience of the process must also be evaluated.

For the process of concentration in general virus purification procedures, literature reveals processes for concentrating and exchanging the solution surrounding the virus that have been applied in various virus purification procedures (Lyddiatt, A. and O'Sullivan, D. A., (1998), Curr. Opin. Biotechnol., 9(2):177-85; Summerford, C. and Samulski, R. J., (1999), Nat. Med., 5(5):587-8; Transfiguracion, J. et al., (2003), Hum. Gene. Ther., 14(12):1139-53), for instance, purification of adenovirus by precipitation with ammonium sulfate (Schagen, F. H. et al., (2000), Gene Ther., 7(18):1570-4) and by ultracentrifugation (Ugai, H. et al., (2005), Biochem. Biophys. Res. Commun., 331(4):1053-60). Furthermore, ultrafiltration can be utilized for concentrating reverse transcript virus, densonucleosisvirus and human influenza A virus (Geraerts, M. et al., (2005), J. Gene Med., 7(10): 1299-310; Grzenia, D. L. et al., (2006), Biotechnol. Prog., 22(5):1346-53; Reiser, J., (2000), Gene Ther., 7(11):910-3; Wickramasinghe, S. R. et al., (2005), Biotechnol. Bioeng., 92(2):199-208). In some literature, effects of filter membrane pore size on the recovery yield of enveloped reverse transcript virus, densonucleosisvirus and human influenza virus are analyzed (Cruz, P. E. et al., (2000), Biotechnol. Prog., 16(3):350-7; Miller, D. L. et al., (1996), Nucleic Acids Res., 24(8):1576-7; Grzenia, D. L. et al., (2006), Biotechnol. Prog., 22(5): 1346-53).

For the purification process in general virus purification procedure, the recovery yield of the adeno-associated virus by ultracentrifugation and affinity chromatography are compared in literature in 2001 (Auricchio, A. et al., (2001), Molecular Therapy, 4(4): 372-374). Further, it is reported that an adeno-associated virus is purified by using an ion exchange method (Chahal, P. S. et al., (2006), J. Virol. Methods, doi: 10.1016/j.jviromet.2006.09.011). Literatures also demonstrate that use of heparin-affinity chromatography and metal ion exchange methods in the purification of reverse transcript virus, which can result in a recovery yield of up to 70% (Segura, M. M. et al., (2005), Biotechnol. Bioeng., 90(4):391-404; Yu, J. H. and Schaffer, D. V. (2006), Journal of Virology, 80(7): 3285-3292). Accordingly, column chromatography is gradually replacing conventional ultracentrifugation to become an optimal option among the techniques for purifying virus vectors.

With respect to the purification procedure for baculovirus, commonly used procedures for purifying baculovirus simply consist of a process of ultracentrifugation (Transfiguracion, J. et al., (2007), J. Virol. Methods, 142(1-2): 21-8). However, ultracentrifugation is time-consuming, unable to be carried out on a large scale and causes damage to the envelope of the virus. Despite the yield of virus by ultracentrifugation being able to be increased by using ammonium sulfate, the increased salt concentration of the resulting virus solution causes loss of virus activity and decreases the recovery yield of viruses with activity (Shi, L. et al., (2005), J. Pharm. Sci., 94(7):1538-51). Dialysis methods have also been utilized to exchange the virus surrounding solution, but this is slow and time-consuming.

Chromatography is also applied in processes for purifying baculovirus and include ion exchange chromatography, gel filtration and affinity chromatography. Cation exchange chromatography is employed to concentrate a baculovirus solution (Barsoum J., (1999), BioTechniques, 26: 834-840); however, there is still a problem of poor purity. A method for purifying baculovirus with a combination of ultracentrifugation and gel filtration, which has a recovery yield of 25% has been disclosed, however, according to the results of SDS-PAGE in the literature, the purity is not satisfactory (Transfiguracion J. et al., (2007), J. Virol. Methods, 142(1-2):21-8).

Presumably, a cause of low recovery yield of baculovirus lies in that the envelope thereof being easily broken down when passing through the column, which leads to lost of activity. Recently, a process for purifying baculovirus by ion exchange membrane has been developed (Wu et al., (2007), Human Gene Therapy, 18: 665-672). Although the process has an improved recovery yield of 78%, the purity of the virus obtained by the process remains poor.

Isolation of the virus by affinity chromatography is based on the binding between viruses and molecules with the specific affinity to the molecules on viral particles. Except for viruses being seldom damaged during the procedure of affinity chromatography, the undesired impurity can be effectively removed and the purity of obtained virus is improved. The applicants have previously developed a method for purifying baculovirus by immobilized metal affinity chromatography (IMAC) with hexahistidine (His6). The method can obtain a purity up to 87%, but with a low recovery yield of 2˜3% (Hu, Y-C et al., (2003), Enzyme Microb. Technol., 33: 445-452).

Based on the aforesaid, current techniques for purifying baculovirus have poor virus recovery yield and purity and are usually time consuming and laborious. Therefore, there exists a need for a novel method for purifying baculovirus with improved virus recovery yield and high efficiency.

### SUMMARY OF THE INVENTION

The main objective of the invention is to provide a method for purifying baculovirus with an increased virus recovery yield and purity of virus.

A method for purifying baculovirus in accordance with the present invention comprises: providing a baculovirus mixture containing a baculovirus and a liquid portion;replacing the liquid portion with a binding buffer by an ultrafiltration system to form a virus buffer; and purifying the baculovirus from the virus buffer with glycoprotein specific affinity chromatography.

In brief, the method of the present invention employees an ultrafiltration system and glycoprotein specific affinity chromatography. The ultrafiltration system is time-saving and can achieve a high recovery yield for replacing virus surrounding solution. On the other hand, the glycoprotein specific affinity chromatography is able to purify baculovirus on a large scale. Therefore, the present invention for purifying baculovirus has the advantages of being able to achieve enhanced virus recovery yield and improved purity of virus as well as being time-saving and convenient.

Other objectives, advantages and novel features of the invention will become more apparent from the following detailed description when taken in conjunction with the accompanying drawings.

### BRIEF DESCRIPTION OF THE DRAWINGS

FIG. 1 illustrates the effect of buffer on the transducing ability of virus;

FIG. 2 illustrates the results of optimization for ultrafiltration; and

FIG. 3 illustrates the results of characterization of affinity chromatography in example 3.

### DETAILED DESCRIPTION OF THE PREFERRED EMBODIMENTS

The present invention relates to a method for purifying baculovirus, comprising: providing a baculovirus mixture containing a baculovirus and a liquid portion; replacing the liquid portion with a binding buffer by an ultrafiltration system to form a virus buffer; and purifying the baculovirus from the virus buffer with glycoprotein specific affinity chromatography.

As used herein, “baculovirus mixture” refers to a mixture containing a baculovirus and a liquid portion, wherein the liquid portion may be any bio-compatible solution, including, but not limited to, cell culture medium, saline, saccharide solution and buffer; preferably, the liquid portion is cell culture medium.

According to the present invention, wherein the baculovirus is a nuclear polyhedrosis virus.

According to the method of the present invention, wherein the baculovirus is selected from the group consisting of Autographa californica nucleopolyhedrovirus (AcMNPV), Bombyx mori nucleopolyhedrovirus (BmNPV), Buzura suppressaria nuclear polyhedrosis virus, Cryptophlebia leucotreta granulosis virus, Lymantria dispar baculovirus, Mamestra brassicae nuclear polyhedrosis virus, Orgyia pseudotsugata mononuclear polyhedrosis virus, Penaeus monodon-type baculovirus, Plodia interpunctella granulosis virus and Trichoplusia ni mononuclear polyhedrosis virus and modified viruses thereof.

Preferably, the baculovirus is selected from a group consisting of Autographa californica nucleopolyhedrovirus (AcMNPV) and modified viruses thereof.

As understood in the art, the term “modified virus” as used herein refers to a recombinant virus having an exogenous gene inserted during the process of cloning.

In a preferred embodiment of the present invention, wherein the baculovirus is vesicular stomatitis virus G protein-modified Autographa californica nucleopolyhedrovirus (VSVG-AcMNPV).

As used herein, “ultrafiltration system” refers to a commonly used filtration technique for replacing, desalting or concentrating. Depending on proteins to be retained, the filter membranes with nominal molecular weight limits in a range of 1 kD to 1000 kD are used, wherein the filter membrane is rated by nominal molecular weight limits (NMWL).

A membrane pore size rating, typically given as a micron value, indicates that particles larger than the rating will be retained by the membrane. A NMWL, on the other hand, is an indication that most dissolved macromolecules with molecular wights higher than the NMWL and some with molecular weight lower than the NMWL will be retained by the membrane.

According to the method of the present invention, wherein the ultrafiltration system includes, but is not limited to, tangential flow filtration (TFF), stirred cell filtration and centricon. In a preferred embodiment of the present invention, tangential flow filtration and stirred cell filtration of normal flow filtration are used for preliminarily concentrating and replacing solution surrounding virus.

In one of embodiment of the present invention, the ultrafiltration system may be tangential flow filtration. Tangential flow filtration uses filter membranes having a surface, a filtration side and pore size are manufactured by piling up multiple membranes to prepare a large scale sample in tangential flow filtration fluid has a tangential flow along the surface of the membrane with a portion of fluid being forced through the membrane to the filtration side by an applied pressure. Particles and macromolecules too large to pass through the membrane pores are swept along by the tangential flow. Therefore, tangential flow filtration prevents retained component building up at on the surface of the membrane and causing gel polarization on the membrane. According to the transducing titers and recovery yield of obtained virus and time needed for performing the two systems described above, the ultrafiltration system for replacing the virus surrounding solution is preferably tangential flow filtration.

According to the method of the present invention, wherein the tangential flow filtration is performed by a constant-volume diafiltration process being recycled ultrafiltration.

According to the method of the present invention, wherein the tangential flow filtration is performed by a filter membrane module consisting of, but not limited to, hollow fiber, spiral-wound and flat plate filter membrane.

According to the method of the present invention, wherein the tangential flow filtration is performed under pressure ranging from 2 to 20 psi; more preferably, the pressure range is from 5 to 10 psi.

Alternatively, the ultrafiltration system is stirred cell filtration. Stirred cell filtration is a type of normal flow filtration using a filter membrane having a surface and multiple pores. In normal flow filtration, fluid is forced to pass through, in a direction normal to the surface of the membrane under an applied pressure. In stirred cell filtration, a stirrer agitates approximately adjacent to the surface of the membrane to avoid a localized high concentration of molecules blocking the pores of the membrane.

According to the method of the present invention, wherein the ultrafiltration is performed with a filter membrane suitable for purifying the baculovirus in accordance with the present invention. Preferably, the filter membrane of ultrafilration has nominal molecular weight limit ranging from 1 to 1000 kDa. Preferably, the filter membrane of the ultrafiltration has nominal molecular weight limit ranging from 10 to 800 kDa. More preferably, the filter membrane of the ultrafiltration has nominal molecular weight limit ranging from 100 to 500 kDa. Most preferably, the filter membrane of the ultrafiltration has nominal molecular weight limit ranging from 200 to 400 kDa.

In a preferred embodiment of the present invention, the filter membrane of the ultrafiltration has a nominal molecular weight limit of 300 kDa.

According to the method of the present invention, wherein replacement of the liquid portion of the baculovirus mixture is performed at a temperature ranging from 10° C. to 40° C.; more preferably, from 15° C. to 35° C.; and more preferably, from 20° C. to 30° C.

According to the method of the present invention, wherein the binding buffer is any buffer adapted for glycoprotein specific affinity chromatography.

Preferably, the binding buffer contains: 20 mM Tris, 0 to 0.5 M sodium chloride (NaCl), 0.5 to 1.5 mM calcium chloride (CaCl₂), and 0.5 to 1.5 mM manganese chloride (MnCl₂).

In one of preferred example of the present invention, wherein the binding buffer is a solution containing 20 mM Tris, 0.5 M NaCl, 1 mM CaCl₂ and 1 mM MnCl₂ at pH 7.4.

Because the baculovirus according to the present invention has glycoprotein on its surface, the method according to the present invention is performed by glycoprotein specific affinity chromatography for purifying baculovirus. As used herein, “glycoprotein specific affinity chromatography” refers to affinity chromatography that is specifically for purification of glycoprotein by using a molecule having specific binding activity to glycoprotein.

The glycoprotein specific affinity chromatography in accordance with the present invention comprises:

contacting a baculovirus solution with a molecule having specific affinity to glycoprotein, whereby baculovirus contained in baculovirus solution binds to the molecule having specific affinity to glycoprotein for absorption;

using a washing solution for washing;

and using a elution solution to elute the baculovirus binding to the molecule specific to glycoprotein affinity to obtain purified virus.

In one of preferred example of the present invention, the molecule having specific affinity to glycoprotein in the glycoprotein specific affinity chromatography is lectin.

In one of preferred example of the present invention, wherein lectin is selected from the group consisting of Con A, Lentil lectin and wheat germ agglutinin (WGA).

According to the method of the present invention, wherein the glycoprotein specific affinity chromatography is performed at a temperature ranging from 15° C. to 35° C.; preferably, from 20° C. to 30° C.

According to baculovirus having gp64 glycoprotein which is able to bind to Concanavalin A (Con A), in one preferred example of the present invention, ConA resins are used to bind to glycoprotein expressed on the surface of the virus. Con A is an abbreviated term for Concanavalin A, a lectin extracted from Canavalia ensiformis, which is a protein synthesized and secreted by animal cells and plant cells and is capable of binding to saccharides. Con A binds to a portion of molecules containing D-mannopyranosyl and D-glucopyranosyl and is generally used to separate and purify glycoproteins, polysaccharides and glycolipids.

In the affinity chromatography, supports which are conjugated to components capable of binding to saccharides are generally gels. The gels may be polydextran, polyacrylamide (IBF Biotech, Ultragel) and argarose. The gels are chemically inert and separation will not induce denature or other chemical reactions. Preferably, the materials of gels have stability for long term use and can be used in a large range of pH values and temperatures. Preferably, ConA is conjugated to argarose.

In one preferred example of the present invention, wherein Con A resins are Con A agarose resins.

In one preferred example of the present invention, wherein Con A resins are Con A Sepharose 4B.Sepharose is a trademark of argarose produced by Sigma-Aldrich, belonging to nature gels with large pore size and Sepharose 2B, 4B and 6B respectively represent Sepharose containing 20%, 40% and 60% of dry gel by weight and are mainly used for preparing substances such as nucleic acids or viruses with a molecular weight over 400,000.

Since particles of gels are soft they tend to plug the column during the process of separation. This results in slowing down flow speeds. Sepharose will melt at a temperature over 50° C., so is required to be used below 50° C. Argarose after being made into beads must not be dehydrated, therefore, must be stored in humid conditions.

Con A Sepharose 4B is applied in purification of enzyme antibody conjugates, immunoglobulin (IgM) and glycosylated protein (Franco Fraguas L. et al., (2004), J. Chromatogr. B. Analyt. Technol. Biomed. Life Sci., 803(2):237-41; Lambotte, O. et al., (2003), J. Rheumatol., 30(5):1027-8; Ray, P. K. and Raychaudhuri, S., (1982), Biomed. Pharmacother., 36(4):206-10). For maintaining the binding activity of Con A Sepharose 4B, Manganese(II) (Mn²⁺) and Calcium(II) (Ca²⁺) were added. Especially when the pH value is lower than 5, these metal ions are required to maintain binding activity of the gels. Therefore, the buffer used in the glycoprotein specific affinity chromatography according to the present invention, such as binding buffer, washing buffer and elution solution, preferably contain Manganese(II), Calcium(II) or both.

In a preferred example of the present invention, wherein the glycoprotein specific affinity chromatography is performed with an elution solution containing saccharides.

According to the method of the present invention, wherein the saccharides of the elution solution are selected from the group consisting mannoside, mannose, glucose, glucoside and derivatives thereof.

Preferably, the elution solution contains mannoside and derivatives thereof. More preferably, the elution solution comprises α-methyl-D-mannoside at a concentration ranging from 0.5 M to 1.2 M.

In another preferred example of the present invention, wherein the elution solution further contains 0.5 M NaCl.

In another preferred example of the present invention, wherein the elution solution contains saccharides and is at pH 7.4.

In another preferred example of the present invention, wherein the glycoprotein specific affinity chromatography comprises a washing step by using a buffer solution containing 20 mM α-methyl-D-mannoside to remove undesired protein to increase virus recovery yield and purity of obtained virus.

The present invention was further illustrated by the following examples; it should be understood that the examples and embodiments described herein are for illustrative purposes only and should not be construed as limiting the embodiments set forth herein.

### EXAMPLES

### General Experimental Materials and Methods

1. Insect Cell Culture

In the following examples, cell line Sf-9 of insect cells Spodoptera frugiperda (Food Industrial Research and Development Institute, Taiwan) was propagated in TNM-FH (Sigma) supplemented with 0.1% Pluronic F-68 (Sigma, Missouri) and 5% fetal bovine serum (FBS) (Gibco BRL) in a spinner flask at 80 rpm. The culturing procedure is according to standard protocol at a temperature of 27° C. (O'Reilly, D. et al. (1992), Baculovirus expression vector: a laboratory manual. Baculovirus expression vector. New York: W.H. Freeman and Co).

2. Mammalian Cell Culture

In the following examples, mammalian cells are human cervical cancer cells (HeLa). The cells were maintained in Dulbecco's Modified Eagle Medium (DMEM) (Sigma) supplemented with 5% FBS at 37° C. in a 5% CO₂ incubator with a 150 cm² flask (T-150 flask). When cell density reaches about 1 to 2×10⁷, the residual medium was washed away by using 10 mL Dulbecco's phosphate buffered saline (DPBS) at pH 7.4 and DPBS was then removed. 3 mL trypsin-EDTA solution (Invitrogen) was added and evenly applied to the cell culture by gentle agitation and the cell culture was then incubated at 37° C. in an incubator for 5 to 10 mins for detaching cells. Cell densities and viability of detached cells were respectively determined by a hemacytometer and trypan blue dye exclusion method (O'Reilly et al., supra). By the cell density, 3×10⁶, cells were inoculated into another T-150 flask and the volume of cell culture was filled up to 20 mL for use.

3. Amplification of Recombinant Baculovirus

In the following examples, the recombinant baculovirus AcVSVG-CAGFP was kindly provided by Prof. Yoshigaru Matsuura (DVM, Japan) and was used to transduce HeLa cells. AcVSVG-CAGFP is driven by polyhedrin promoter to express VSVG. It is known in the art that the expression of VSVG glycoprotein on the surface of viral particle can enhance the transduction efficiency in cells (Mangor, J. T., et al. (2001), J. Virol., 75(6): 2544-56). To amplifying viruses, Sf-9 cells were cultured in 200 mL spinner flask until the cell density reached 1×10⁶ cells/mL. The culture was centrifuged at 1000 g for 10 mins, followed by removal of the supernatant. An obtained cell pellet was resuspended in 20 mL fresh medium, and recombinant baculovirus was added at an infecting viral dosage of 0.1 MOI (multiplicity of infection), followed by being sufficiently mixed for 1 hour. After, the mixture of the cells and virus was placed in a spinner flask, filled up with 180 mL fresh medium and incubated at 27° C. in an incubator at a speed of 80 rpm. During infection, cell growth and cell viability were observed. Once the cell viability fell to 70%, cells and cell debris were removed by centrifuging at 3,000 g for 10 mins. Obtained supernatant was hereafter a virus solution and stored at 4° C. in the dark.

4. Transduction of Mammalian Cells by Baculovirus

In the following examples, transduction of mammalian cells is performed according to the transduction procedure previously developed by the applicants (Chan, Z. R. et al., (2006), Biotechnol. Bioeng., 93(3):564-71). In the transduction procedure the process of concentrating the virus by ultracentrifugation is absent, which not only reduces loss of virus but also simplifies the transduction procedure and enhances the virus transduction efficiency by providing more options for surrounding solution. During the transduction, cells were seeded into 6-well plates and incubated at 37° C. in a 5% CO₂ incubator. When cells were attached after about 12 hours, culture medium was removed and the cells were washed with DPBS to remove the residual culture medium, followed by an addition of 100 μL of unconcentrated virus solution and 400 μL DPBS to adjust a final volume to 500 μL. The plates were slowly shaken on a shaking plate at 25° C. After 6 hours, the mixture of virus and DPBS was aspirated and replaced with 2 mL of culture medium, followed by incubation at 37° C. in a 5% CO₂ incubator to finish the transduction procedure.

5. Flow Cytometry Analysis

A. Determination of Transduction Efficiency by Flow Cytometer

After HeLa cells were transduced and incubated at 37° C. in a 5% incubator for one day, the cells were detached with trypsin-EDTA, resuspended in DPBS prefiltered with 0.22 μm filter (1 mL/well), and then placed into a vial specific for flow cytometer (BD Falcon™ 352052) for flow cytometry analysis (FACSCalibur, Becton Dickinson). A percentage of cells emitting fluorescence (% GFP⁺ cells) and mean fluorescence intensity (FI) (defined as arbitrary units, au) were calculated as the mean value of three measurements by counting 10,000 cells.

B. Determination and Calculation of Transducing Titers

Transducing titer (TT), a unit for quantifying an ability of baculovirus to transduce into mammalian cells, was determined by a method called “transducing titration” (Chan Z R et al. (2006), supra). The virus solution was serially diluted twofold in TNM-FH medium. Virus solutions diluted by different dilution factors were used to transduce HeLa cells as described above. At 24 h post-transduction, the transduction efficiency and mean fluorescence intensity for HeLa cells were determined by flow cytometry. Transducing titers were calculated according to the following equation (Logan, A. C. et al., (2004), Hum. Gene Ther. 15(10):976-88; Transfiguracion, J. et al., (2004), J Chromatogr B Analyt Technol Biomed Life Sci, 813(1-2):167-73), and were expressed as transducing units per milliliter (TU/mL):

Transducing titer (TU/mL)=[(% GFP⁺ cells/100)](TU)×dilution factor×cell number×reciprocal of volume (mL⁻¹)

Noting that the transducing titer was calculated based on the first dilution factors that resulted in fewer than 20% of GFP⁺ cells and the value of transduction efficiency thereof. If the virus transducing ability was so low that the transduction efficiency was below 20% without any dilution, the dilution factor was set at 1. A virus transducing dose unit with similar definition to MOI was derived as multiplicity of transduction (MOT) and expressed as TU/cell.

C. Quantitation of Viral Particles by Flow Cytometry

New protocols for quantifying viral particles by using flow cytometry were gradually developed in this decade (Brussaard, C. P. et al., (2000), J. Virol. Methods, 85(1-2): 175-82; Marie, D. et al., (1999), Appl. Environ. Microbiol., 65(1):45-52). Recently, a literature was aimed at baculovirus to develop a protocol for quantifying viral particles (Shen, C. F. et al., (2002), J. Virol. Methods, 105(2):321-30), which is a direct count of a physical viral particle number of viruses containing DNA. Virus solution was filtered through a 0.45 μm filter membrane (Sartorius), and then diluted with DPBS (pH 7.4, filtered through a 0.22 μm filter membrane) to about 10⁶ viral particles/mL (in general condition, about 10² to 10⁴ fold). Each 950 μL of diluents was added to 20 μL of 5% paraformaldehyde solution (Sigma) (prepared by dissolving and heating powder in DPBS at pH 7.4 at 60° C. followed by filtration through a 0.22 μm filter membrane and being stored, exceeding one week, at 4° C. until use). The resulting solution chilled to 4° C. for 45 mins and further chilled at −80° C. for at least 10 mins, before being thawed in a water bath at room temperature. Subsequently, 10 μL of 10% Triton X-100 (Sigma) prepared by diluting with pH 7.4 DPBS and filtering through a 0.22 μm filter membrane) and added and agitated to mix homogenously, followed by standing for 5 mins, adding 20 μL SYBR Green I dye (Molecular Probes Inc. Eugene, Oreg.) (10,000-fold concentrated and diluted 200 fold with pH 7.4 DPBS before use) and being stored in the dark for 10 mins. After being placed on ice to chill for 10 mins, the obtained solution was transferred into a vial for flow cytometer (BD Falcon™ 352052) and analyzed by flow cytometry with a band filter of 530 nm (FACSCalibur, Becton Dickinson).

6. Protein Analysis

A. SDS-PAGE

SDS-PAGE was performed according to Laemmli method (Laemmli et al., (1970), J. Mol. Biol., 47(1): 69-85). Protein samples were prepared and analyzed by SDS-PAGE electrophoresis device produced by Bio-Rad cooperation. Gels were prepared by protein electrophoresis device (Bio-Rad). In the following examples, gels composed of 12% running gel and 5% stacking gel (formula as shown in table 1) were used. After being obtained, gels were set up in a tank and running buffer (25 mM Tris, 250 mM glycine, 0.1% SDS, pH 8.3) was added. Protein samples were mixed with 4× sample buffer (3.75 mL 1 M Tris-HCl (pH=6.8), 1.2 g SDS, 4 mg bromophenol blue, 4 mL glycerol, 1.5 mL β-mercaptoethanol) in a ratio of 1 to 3 (depending on the formula used) and placed at 95° C. The protein samples were injected into each well of gels. The electrophoresis was performed at a voltage of 70 V for 20 mins. Voltage was increased to 110V for 1 hour when dye entered into the running gel. Gels were analyzed when the dye approached the bottom of the gels.

TABLE 1
SDS-PAGE gel composition

| 12% running gel (5 mL) | | 5% stacking gel (1 mL) | |
|---|---|---|---|
| | volume | | volume |
| composition | (mL) | composition | (mL) |
| ddH₂O | 1.6 | ddH₂O | 0.68 |
| 30% acrylamide mix | 2 | 30% acrylamide mix | 0.17 |
| 1.5 M Tris (pH = 8.8) | 1.3 | 1 M Tris (pH = 6.8) | 0.13 |
| 10% SDS | 0.05 | 10% SDS | 0.01 |
| 10% ammonium | 0.05 | 10% ammonium persulfate | 0.01 |
| persulfate | | | |
| TEMED | 0.002 | TEMED | 0.001 |

B. Western-Blot

Obtained SDS-PAGE gel was blotted to nitrocellulose membrane (NC membrane) by Mini Trans-Blot Cell (Bio-Rad). NC membranes and gels containing protein samples were immersed in transfer buffer (25 mM Tris, 192 mM glycine, 20% methanol, pH 8.3). Followed by sandwiching between filter papers, the gel was placed on the negative pole and overlaid with the NC membrane. Finally, aforesaid gel assembly was placed between sponges and was prepared for transfer. The transfer took place at a voltage of 100V for 60 mins. After the transfer was accomplished, the NC membrane was immersed in blocking buffer (5% defat milk powder in 25 mM Tris, 150 mM NaCl, 0.05% Tween-20; pH=7.4) at room temperature for 1 hour or at 4° C. overnight. Subsequently, the NC membrane was washed with TBST 3 times, each time for 5 mins. The NC membranes were respectively incubated in solutions containing primary antibodies of gp-64 (by dilution factor of 1:1000, eBioscience) and vp-39 (by dilution factor of 1:2000, kindly provided by Prof. Loy Volkman, UC Berkeley, USA) at room temperature for 1 hour or at 4° C. overnight. After being washed with TBST three times, the NC membranes were incubated in blocking buffer added with secondary antibodies (by dilution factor of 1:3000, conjugated with AP) (KPL) and allowed to react at room temperature for 1 hour. The NC membranes were washed with TBST three times and developed with 1 mL BCIP/NBT (Sigma) for analysis.

C. Protein Assay

Protein assay was performed by Protein Assay Kit (Bio-Rad). BSA with known concentration (10 μg/mL) was diluted 1.25, 2.5, 5, 7.5 and 1.0-fold as standards for the present assay. 400 μL of each standard and 100 μL dye (Protein Assay Dye Reagent Concentrate, catalog number 500-0006, Bio-Rad, USA) were mixed evenly and left to stand for 10 mins. The absorption was measured at a wavelength of 595 nm. A standard curve was generated by plotting obtained absorbance values against concentration. In the following examples, fractions obtained from each step of purification including loading (Load), flowthrough (FT), washing (wash) and elution (E) were diluted as described above and their absorbance was further measured at a wavelength of 595 nm. The obtained protein concentration was calculated using the interpolation method.

7. Transmission Electron Microscopy (TEM)

Samples were pretreated with processes of fixation, dehydration, infiltration, embedding and staining. The present example employed negative staining method.

Carbon coated copper grid (Argar, England) was placed on a parafilm. 5 μL centrifuged virus solution was placed on the copper grid and left to stand for 1 minute, followed by removing excess water with filter papers. 5 μL dye (2% phosphotungstic acid, PTA, pH 7.4) was droppered onto the copper grid and after being left to stand for 1 minute, excess dye was withdrawn by filter papers. After being dried, the sample was observed by electron microscope (Field Emission Gun Transmission Microscope with Energy Dispersive X-Ray Spectrometer, 300 kV, Expensive Device Center, NTU, Taiwan).

8. Statistical Analysis

Data from at least 3 repeated experiments was used in value difference analysis. Student's t test was used to calculate a p value, where p values less than 0.05 were considered statistically significant.

### Comparison Example 1

### Purification of Baculovirus by Centrifugation

To develop a novel means for purifying viruses, the present comparison example was carried out for realizing disadvantages of the conventional means for purifying virus. In the present comparison example, baculovirus was purified by ultracentrifugation as control and the purified virus was transduced into HeLa cells to determined the virus titers by methods as described in “General experimental materials and methods”. The virus recovery yield was determined by the virus titers before and after purification.

### Experimental Method:

The majority of methods for concentrating and purifying insect baculovirus require ultracentrifugation (O'Reilly et al. (1992), supra) repeated three times. The first centrifugation was for concentrating. 30 mL virus solution was separated into 3 centrifuge tubes and 1 mL 25% sucrose solution in PBS was added at the bottom of the centrifuge tubes by using a syringe with long needle for reducing damage to the virus due to centrifugation. Subsequently, the centrifuge tubes were centrifuged at 80,000 g for 1.5 hours to precipitate the virus. The supernatant was then removed and the virus pellet was resuspended in 3 mL PBS. The second centrifugation was for purifying virus. Obtained virus suspension was subjected to gradient ultracentrifugation (90,000 g, 3 hours) by using 35, 43, 55 and 60% (wt/wt) sucrose solution. The virus pellet obtained by ultracentrifugation was located at a position corresponding to about 48% sucrose and was drawn out with a needle. The third centrifugation was for replacing the sucrose solution with PBS. Purified virus solutions were stored at −80° C. or 4° C.

### Results:

According to SDS-PAGE result, samples obtained by ultracentrifugation contained a band corresponding to 64 kDa, confirming the existence of the major envelope protein gp64 of the virus. Furthermore, a band corresponding to 39 kDa confirmed the existence of the nuclear protein vp39 of the virus. The observation confirmed the existence of envelope protein and nuclear protein to ascertain that an intact virus was obtained. Comparing to the protein content of virus solutions before and after ultracentrifugation, the purified virus sample showed decreased noise, indicating that impurity was effectively removed by using ultracentrifugation to purify viruses such that the purity was increased.

Table 2 shows that virus total titer prior to purification was 1.88×10⁹ TU, so a level of 3.59×10⁶ after purification represents a 0.19% virus recovery yield. Presumably, ultracentrifugation resulted in a breakdown of the virus or virus aggregation, which led to low recovery yield.

TABLE 2
Recovery yield for virus titer before and after purification by
ultracentrifugation

| | | | Total transducing | |
|---|---|---|---|---|
| | Volume | | titers | Recovery |
| Fractions | (mL) | TU/mL | (Total TU) | yield (%) |
| Before | 30 | 6.27 × 10⁷ | 1.88 × 10⁹ | 100 |
| After | 1 | 3.59 × 10⁶ | 3.59 × 10⁶ | 0.19 |

Although purification of viruses by ultracentrifugation increased purity, the recovery yield was very low. Therefore, it is considered to establish a novel method for purifying viruses to retain a high purity and enhance recovery yield.

### Example 1

### Analysis of Transducing Ability of Baculovirus in Various Solutions for Chromatography

Prior to affinity chromatography, the virus surrounding solution was replaced with buffer suitable for chromatography to enhance the efficiency for filtrating impurities to reduce impurities in medium. The condition of the used buffer influenced binding between the virus and resins as well as the elution efficiency. The present example was principally aimed at characterizing optimal buffer conditions for baculovirus to prevent loss of activity under an improper environment, this affected results of purification.

The present example investigated the effects of basal buffer, i.e. 20 mM Tris-buffer (20 mM Tris-hydrochloric acid (HCl), 1 mM CaCl₂,1 mM MnCl₂), with various concentrations of saccharides, concentrations of salts or pH values on virus transducing abilities.

### Experimental Methods:

In the present example, virus solutions were concentrated to the desired virus titers. 100 μL of virus solution was added into an eppendorf and 900 μL of buffer having different compositions as described in Table 3 was subsequently added according to conditions of chromatography solution as desired. The conditions for comparison are shown in Table 3 as the follows:

TABLE 3
Buffer conditions

| | | | | | | Fixed |
|---|---|---|---|---|---|---|
| | Buffer conditions adjusted | | | | | conditions |
| mannoside | 0 M | 0.25 M | 0.5 M | 0.75 M | 1.0 M | 0.5 M NaCl |
| | | | | | | pH 7.4 |
| NaCl | 0.1 M | 0.3 M | 0.5 M | 0.75 M | | pH 7.4 |
| pH | 6.2 | 6.8 | 7.4 | 8.0 | | 0.5 M NaCl |

Condition of Basal buffer:
20 mM Tris-HCl, 1 mM CaCl₂, 1 mM MnCl₂

For simulating conditions after purification, a mixture of virus and buffer as described above was placed at room temperature for 12 hours in the dark. The obtained virus mixture was filtrated through a 0.45 μm filter membrane and was transduced into HeLa cells. For 24 hours after transduction, cells were analyzed by flow cytometer for their transducing efficiency and fluorescence intensity. Last, the total fluorescence intensity was calculated, and was used to determinate the optimal binding buffer for chromatography.

In addition, to confirm to differences of transducing efficiency resulting from the purification procedure but not the osmolarity of the buffer itself, the osmolarity of used buffer was determined by vapor pressure osmometer (MODEL 5520, WESCOR) according to the measuring method provided by the manufacturer.

### Results:

1. The Effect of Concentration of Saccharides on Baculovirus:

Purification of virus by Con A resin affinity chromatography generally required addition of mannoside to compete and elute the virus. Therefore, it was necessary to determine an effect of concentration of saccharides during the purification procedure on virus transducing ability. To coordinate the conditions of the subsequent steps in the purification procedure, in addition to the experiment being performed under room temperature in the dark for 12 hours, buffers (20 mM Tris-HCl, 0.5 M NaCl, pH 7.4) supplemented with 0.25 M, 0.5 M, 0.75 M and 1.0 M mannoside were used in the present example. The obtained results were further compared to those using 0 M mannoside buffer to evaluate an effect of the concentration of mannoside to the virus transducing ability.

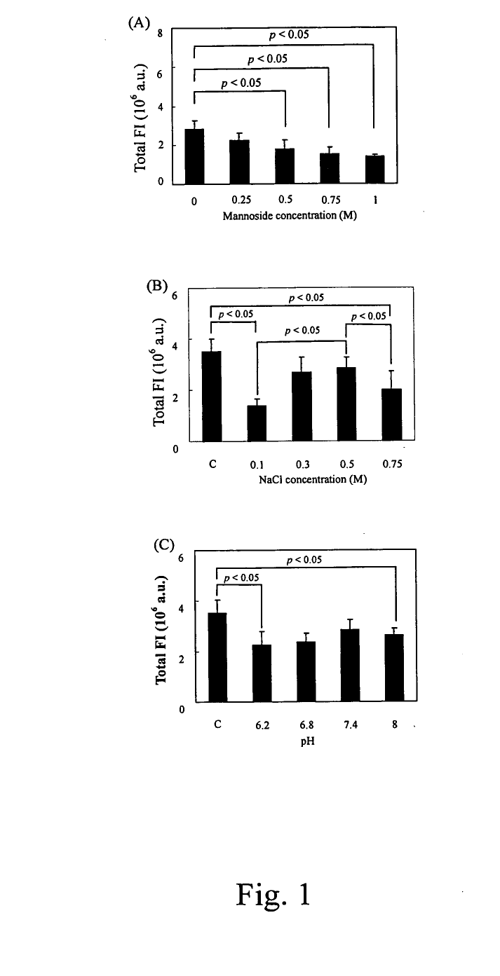

With respect to an effect of the composition of buffer on virus transducing ability, FIG. 1A indicates that the total fluorescence intensity of HeLa cells transduced with 0 M mannoside buffer was 2.8×10⁶ au. These results show that total fluorescence intensity declined with an increase in concentration of the saccharide. The results of statistical analysis showed if the concentration of mannoside was beyond 0.5 M, the total fluorescence decreased to 1.4×10⁶ au, that is 50% of virus stored in 0 M mannoside buffer.

Presumably this can be explained by the following:

(1) saccharides in the solution would increase the osmolarity: As shown in table 4, the osmolarity was about 948 mOsm/L for 0 M mannoside buffer and 2482 mOsm/L for 1 M mannoside buffer. Excess high osmolarity would lead to damage of the virus envelope; and

(2) Used buffer affected cells so as to reduce the transducing efficiency. To reduce damage to cells by dilution, the virus solution was adjusted with PBS in a ratio of 1:4 before being used in transduction. Therefore, there was little possibility that the decrease of virus total fluorescence intensity derived from the effect of the buffer.

TABLE 4
The osmolarity in various conditions of 20 mM Tris-buffer

| | Osmolarity | | Osmolarity |
|---|---|---|---|
| samples | (mOsm/L) | samples | (mOsm/L) |
| pH 7.4, 0.1 M NaCl | 228 | pH 7.4, 0.5 M NaCl | 948 |
| pH 7.4, 0.3 M NaCl | 601 | pH 8.0, 0.5 M NaCl | 954 |
| pH 7.4, 0.5 M NaCl | 948 | 0.25 M mannoside | 1279 |
| pH 7.4, 0.75 M NaCl | 1300 | 0.5 M mannoside | 1634 |
| pH 6.2, 0.5 M NaCl | 1021 | 1 M mannoside | 2482 |
| pH 6.8, 0.5 M NaCl | 950 | Medium only | 369 |

To summarize, the osmolarity of the elution solution increased with the increase of the concentration of saccharides, which led to an excessive osmolarity and affected viability of the virus. The total fluorescence intensity in cells therefore, obviously decreased. Accordingly, if the concentration of overall saccharides was above 0.5 M, the used mannoside-containing solution should be diluted before transduction in order to prevent cells from the damage by the mannoside-containing solution in excessive concentration.

2. Effect of Salt Concentration on Baculovirus

To determine the effect of salt concentration on the virus activity, basal buffer (20 mM Tris-HCl, 1 mM Magnesium Chloride (MgCl₂) and 1 mM CaCl₂, pH 7.4) supplemented with NaCl at concentrations of 0.1 M, 0.3 M, 0.5 M and 0.75 M were respectively mixed with the virus at room temperature in the dark for 12 hours, followed by transducing into HeLa cells. The total fluorescence intensity of the transduced cells was measured. FIG. 1B showed that the total fluorescence intensity of HeLa cells transduced by VSVG-modified baculovirus in original surrounding solution (medium) was 3.5×10⁶ a.u. In a low salt concentration of 0.1 M NaCl, the total fluorescence intensity decreased to 1.4×10⁶ a.u. This suggested that salt concentration would lead to damage of the virus envelope affecting activity and low salt concentration tends to cause virus aggregation resulting in lost of virus activity. These results were consistent with those previously reported (Segura, M. M. et al., (2005), Biotechnol. Bioeng., 90(4):391-404). As shown in Table 4, when the salt concentration was increased to 0.75 M NaCl, the osmolarity of medium altered from 369 mOsm/L to 1300 mOsm/L. The altered osmolarity due to the salt concentration is the major cause behind hampered activity of virus. As virus in buffer with 0.3 M and 0.5 M NaCl did not show a significant decrease in the total fluorescence intensity (as shown in FIG. 1B), indicating that the activities of virus in buffers with those two salt concentrations were higher than those in buffer with 0.1 M and 0.75 M. Consequently, 0.5 NaCl was chosen to be the salt concentration of the buffer for the following steps of purification procedure as suggested in the manual provided by the manufacturer.

3. The Effect of pH Value on Baculovirus:

As described above, in order to determine the effect of pH value on virus activity, buffer (20 mM Tris-HCl, 0.5 M NaCl, 1 mM MgCl₂ and CaCl₂) was adjusted to pH 6.2, 6.8, 7.4 and 8.0 with 1 M HCl or sodium hydroxide (NaOH) and mixed with the virus at room temperature in the dark for 12 hours, followed by being transduced into HeLa cells. Then, the total fluorescence intensity of the transduced HeLa cells were measured. As shown in FIG. 1C, the total fluorescence intensity of HeLa cells transduced with virus in original surrounding solution (medium) was 3.5×10⁶ a.u., while that of HeLa cells transduced with virus buffer with pH 6.8 and pH 6.2 respectively decreased to 2.4×10⁶ a.u. and 2.3×10⁶ a.u. This indicated that an acidic environment had a negative effect on the viability of baculovirus, which differed from the fact that virus are suitable to be preserved in a condition of pH 6.2. It is suggested that the surrounding solution in the present example contained a metal ion (20 mM Tris-HCl, 0.5 M NaCl, 1 mM MgCl₂ and 1 mM CaCl₂) different from the surrounding solution used in the previous experiment, such as medium or PBS. Besides, the different conditions of experiments might lead to deviations of the experiments. After the transduction with buffer at pH 7.4, the total fluorescence intensity of HeLa cells was not significantly altered. Table 4 showed that osmolarity of the buffer was not significantly altered as pH value of the buffer was changed. The osmolarity of the buffer was retained at about 950 mOsm/L and the virus activity was not affected. Therefore, pH 7.4 was chosen as the condition of buffer in the subsequent experiments. As described above, the results indicated that 0.5 M NaCl and pH 7.4 were the optimal conditions for buffer. Therefore, buffer with 20 mM Tris-HCl, 0.5 M NaCl, 1 mM MgCl₂ and 1 mM CaCl₂ at pH7.4 was used in the following examples as binding buffer for chromatography.

### Example 2

### Establishment of Ultrafiltration System

The present example was mainly aimed at establishing a method for replacing the solution surrounding the virus. The method required substituting the solution surrounding the virus by ultracentrifugation by using a suitable binding buffer to facilitate subsequent chromatography in the whole purification procedure, so as to preliminarily filtrate impurities and replace solution as well as to retain high recovery yield. The effects of stirred cell filtration, tangential flow filtration and filter membrane with various pore sizes or nominal molecular weight limits on virus recovery yield and virus activities were evaluated by analysis of the virus transducing titers and virus particles during the whole procedure. The present example also compared the time required for stirred cell filtration and tangential filtration system. Furthermore, a method for replacing virus surrounding solution by using ultrafiltration was established according to the results of the present example.

### Experimental Method:

1. Using Filter Membrane with Various Pore Sizes in Stirred Cell Filtration System

In the present example, filter membranes with a suitable pore sizes were selected according to the particle size of virus according to the present invention. The effect of filter membrane in nominal molecular weight limits of 100 kDa, 300 kDa and 500 kDa on the virus recovery yield was explored by using a stirred, cell filtration system.

As used herein stirred cell filtration system (Stirred cell, Millipore) was performed by using a magnetic stir bar in a filtration container and filtrating the virus solution with buffer. The filter membrane used was BiomaX PB Ultrafiltration Discs (with nominal molecular weight limits of 100, 300 and 500 kDa, Millipore). Prior to use, filter membranes were immersed in DI water, and the water was changed once every 10 minutes three times and then left to stand at 4° C. overnight. 40 mL virus solution and 360 mL binding buffer (20 mM Tris-HCl, 0.5 M NaCl, 1 mM MgCl₂ and 1 mM CaCl₂, pH 7.4) were mixed and added to a stirred cell filtration system set at an average pressure of 5 to 10 psi at room temperature in the dark and at a speed of 40 rpm. The final volume of solution containing virus was set up to be 40 mL. Virus was retained on the membrane and impurities smaller than the corresponding pore size were filtered through, such the solution surrounding the virus was replaced with binding buffer to preliminarily filtrate and replacing the surrounding solution.

2. Tangential Glow Filtration System (TFF System)

The condition of tangential flow filtration was substantially the same as the stirred cell filtration system. The pore size (or nominal molecular weight limits) of filter membranes was selected according to the results of stirred cell filtration system as described above.

A tangential flow filtration system including a tubing pump, device container and filter membrane was used, wherein used filter membrane was Pellicon XL 50 cassettes (with a area of 50 cm², 300 kDa, Millipore). The system was washed with 500 mL ddH₂O and equilibrium was achieved with 500 mL binding buffer, then 40 mL virus solution and 360 mL binding buffer were added and blended. The system was set at an average pressure of 5 psi and kept at room temperature in the dark. The final volume of obtained buffer was set to return to 40 mL, such that the solution surrounding the virus was replaced with binding buffer to obtain a buffer containing virus (virus buffer) due to preliminary filtration and replacement of solution.

### Results:

1. The Effect of the Pore Size of the Filter Membrane on the Recovery Yield

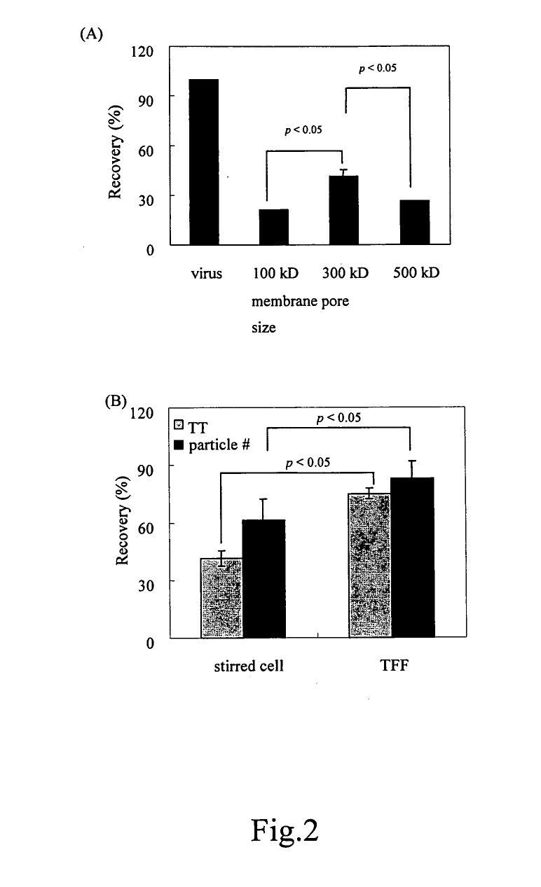

As shown in FIG. 2A, the recovery yield of virus for 300 kDa filter membrane (about 40%) was better than those for 100 kDa and 500 kDa (less than 40%). Presumably, the flux of 500 kDa filter membrane was twice that of 300 kDa filter membrane due to the larger pore size of 500 kDa filter membrane (data not shown). Therefore, viruses easily adhered to the filter membrane resulting in deterring the recovery yield. While using 100 kDa filter membrane, the required time was longer than that of 300 kDa by about 1.5 times (data not shown). This tended to cause gel-polarization on filter membrane to form a gel affecting the recovery yield (Kuiper M. et al., (2002), Biotechnol. Bioeng., 80(4):445-53). Therefore, 300 kDa filter membrane was selected as an optimal filter membrane used in the present invention.

2. The Effect of Various Ultrafiltration on the Recovery Yield

As described above, replacing the solution surrounding the virus by stirred cell filtration system was more convenient than the conventional ultracentrifugation; however, the recovery yield was merely 40%. As shown in FIG. 2B, when tangential flow filtration system was employed to replace the virus surrounding solution, the recovery yield of the virus was increased to 75%, compared to 40% by stirred cell filtration system. Further, the advantages for using tangential flow filtration system were beyond the abovementioned. The recovery yield of viral particles was about 80%. Accordingly, the recovery yield of viral particles and virus titers were not much different, indicating that the virus activity had not been damaged during the process for replacing the solution surrounding the virus. In addition, the experiment using stirred cell filtration system to obtain a 40 mL virus buffer required 150 mins. On the other hand, the tangential flow filtration system only required about 30 mins. Therefore, tangential flow filtration was 5 times faster than stirred cell filtration.

To summarize, the tangential flow filtration system was able to increase the virus recovery yield without decreasing the virus activities. Moreover, it was able to prominently reduce the spent time. Thus the tangential flow filtration system was employed in the following examples for the replacement of the solution surrounding the virus.

### Example 3

### Characterization of the Condition of Affinity Chromatography

The present example was aimed at predetermining the optimal temperature and resins to be used in affinity chromatography before the affinity chromatography was performed.

### Experimental Methods:

For ease of experiments, the present example was performed in an eppendorf. The test for characterizing the condition of affinity chromatography for virus buffer obtained from ultrafiltration system was carried out under the conditions either of the same resin (Con A) and different temperatures (room temperature and 4° C.) or of the same temperature and different resins (Con A and Lentil lectin). Further, binding efficiency between virus and resin at each portion during chromatography was evaluated by virus transducing titers.

The test for characterizing the condition of affinity chromatography was performed as following:

100 μL of resin previously washed with 10 column volume (C. V.) of binding buffer was added to an eppendorf; and then 1 mL of virus buffer obtained by ultrafiltration was loaded into the eppendorf, followed by being evenly agitated by a rotator (Intelli-mixer, ELMI Ltd) for 1 hour, for absorption, and further being centrifuged to obtain a supernatant (i.e. flow-through, FT) for analysis. 1 mL elution solution (containing 0.6 M and 1.0 M mannoside elution solution per mL) was added for elution (E), and centrifuged at 6,000 g for 1 minute. The supernatant was collected as the elution fraction containing virus. Each fraction collected from each step described above was filtered through a 0.45 μm filter membrane and transduced into HeLa cells by the method as described in “General experimental materials and method”, followed by determination of transducing efficiency by flow cytometer. The transducing titers were calculated according to the equation established in the reference (Chan, Z. R. et al., (2006), Biotechnol. Bioeng., 93(3):564-71). The virus recovery yield was calculated based on obtained results from the transducing titer of the originally loaded virus buffer to determine the optimal temperature and resin to be used in the affinity chromatography.

### Results:

1. The Effect of Temperature on Affinity Chromatography

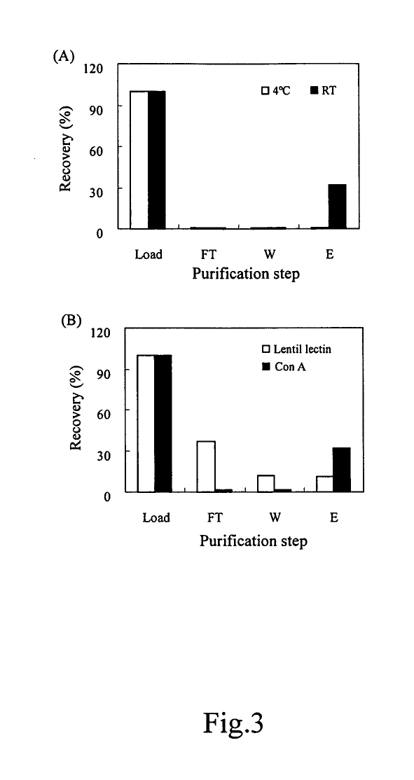

Virus buffer obtained by ultrafiltration was loaded into an eppendorf for test. FIG. 3A shows the binding ability of resin to virus fairly well, whereby little virus was eluted by washing with binding buffer. This was in good compliance with expectations. Applicants noted that virus bound to resin was unable to be eluted with elution solution at 4° C., however, 30% virus was stripped off resins at room temperature. Therefore, 4° C. was not a proper purification condition for obtaining a better virus recovery yield. In the subsequent purification processes in affinity chromatography, the steps for binding and elution of virus and resins were performed at room temperature.

2. The Effect of Resins on Affinity Chromatography

As shown in FIG. 3B, for the results from fraction of flow through (FT), virus and Con A retained a high binding activity resins at room temperature compared to lentil lectin had a low binding activity to virus (about 40% virus fails to bind to resins). For obtaining a better recovery yield, due to the low binding activity between lentil lectin and virus, Con A resins for their superior affinity to virus were employed in affinity chromatography of the subsequent purification procedure.

### Example 4

### Purification of Baculovirus by Affinity Chromatography

### Experimental Methods:

A XK-16 column (Pharmacia Biotech) with a diameter of 16 mm and a height of 20 cm was set up on a vertical iron shelf at room temperature. The column was packed with about 4 mL Con A Sepharose 4B (Amersham Bioscience), and washed and equilibrated by 10 C.V. of binding buffer (20 mM Tris-HCl, 0.5 M NaCl, 1 mM MgCl₂ and 1 mM CaCl₂, pH 7.4). Virus buffer obtained by ultrafiltration was loaded into the column at a speed of 0.3 mL/min for binding of virus to resins for absorption. Flow-through was obtained for analysis. 25 mL each of the binding buffer and the binding buffer supplemented with 20 mM mannoside was subsequently used to wash resins as washing. A fraction of washing was obtained for analysis. Finally, 15 mL and 5 mL of 0.6 and 1.0 M mannoside buffer (α-methyl-D-mannoside in solution with 20 mM Tris-HCl and 0.5 M NaCl) were respectively employed to elute virus at a flow rate of 0.3 mL/min. Eluted virus was diluted one fold with the binding buffer to reduce damage to the virus, followed by being filtered through a 0.45 μm filter membrane and transduced into HeLa cells. Finally, transducing efficiency was determined by flow cytometer and calculated as described above. The virus discovery yield was calculated based on the transducing titer of originally loaded virus buffer. In addition, fractions obtained from aforesaid steps were analyzed by the method as described in “6. Protein analysis” and “7. Transmission Electron Microscopy” in “General experimental materials and methods”. Used resins (UR) after the purification procedure were also subjected to protein analysis for examining if virus remained to bind to resins.

### Results:

After virus surrounding solution was replaced with binding buffer by ultrafiltration and formed a virus buffer, obtained virus buffer was loaded into the column at a flow rate of 0.3 mL/min at room temperature. As shown in Table 5, there was a great discrepancy in the virus titer of the flow through, about 5.27×10⁶ TU, and the virus titer of the fraction prior to being loaded into chromatography column, about 3.78×10⁸ TU. There were two possible causes of the results: First, the strong binding between the virus and resins in the column, so only a proportionately small amount of virus passed through the column; second, the virus was subjected to damage to lose activity while passing through the column, resulting in its low transducing efficiency.

TABLE 5
The recovery yield of baculovirus in each of the steps during
the affinity chromatography after ultrafiltration.

| | | | | Recovery (%) | | |
|---|---|---|---|---|---|---|
| | | | | | | Total viral |
| | | | | | Viral | particle |
| | Volume | Total titers | Total viral | | particles | number/ |
| Fractions | (mL) | (TU) | particles | Titers | number | Total titer |
| Load | 30 | 3.8 × 10⁸ | 1.5 × 10¹⁰ | 100 | 100 | 39.7 |
| Flow through | 30 | 5.3 × 10⁶ | 3.1 × 10⁹ | 1.4 | 20.5 | 588.2 |
| Wash 1 | 25 | 2.3 × 10⁶ | 4.5 × 10⁸ | 0.6 | 3.0 | 199.1 |
| Wash 2 | 25 | 6.9 × 10⁶ | 3.1 × 10⁷ | 1.8 | 0.2 | 4.5 |
| (20 mM mannoside) | | | | | | |
| Elution 1 | 10 | 5.8 × 10⁷ | 2.4 × 10⁹ | 15.4 | 16.0 | 41.2 |
| (pH 7.4, 0.6 M mannoside) | | | | | | |
| Elution 2 | 5 | 2.3 × 10⁷ | 2.0 × 10⁹ | 5.9 | 13.3 | 88.9 |
| (pH 7.4, 1.0 M mannoside) | | | | | | |
| Yield (Total) | 15 | 8.1 × 10⁷ | 4.4 × 10⁹ | 21.3 | 29.3 | 54.5 |

For unveiling the underlying causes, the existence of nuclear protein vp39 and envelope protein gp64 in the flow through was detected by western blotting. In the experiment for detection of vp39 and gp64 proteins, the presence of the two proteins were confirmed by the existence of bands respectively corresponding to vp39 and gp64 proteins. Flow through indeed contained virus proteins but had low transducing efficiency, demonstrating that the virus was damaged so as to lose its activity after passing through the column.

Therefore, for determining the amount of virus damaged during passing through the column, applicants further analyzed viral particle number in the flow through. If the recovery yield of viral particles was obviously higher than the recovery yield of virus titers, virus activity was hampered. The amount of damaged virus could be determined by the two recovery yields. As shown in Table 5, the recovery yield of viral particles in flow through was about 20%, suggesting that approximate 20% of virus was damaged when passing through the column. This might result from the strong affinity between virus envelope protein and resins to cause the lost of activity and then affect the recovery yield.

To summarize, the results of recovery yields of virus titers and viral particles and western blotting demonstrated that virus was slightly damaged during passing through the column by the exceedingly strong binding between the virus and resins. However, the majority of virus (about 80%) still bound to the resins.

Based on the results described above, binding buffer was further loaded to wash off the unbound impurities to increase the purity. In addition, low concentration of saccharides buffer (20 mM mannoside) was loaded to the column to wash off the virus that incompletely bound to resins or impurities bound to resins with low affinity.

As shown in Table 5, the recovery yields of virus titers and viral particles in the fraction obtained from the washing step were respectively 0.60% and 1.81%, demonstrating that only a little amount of virus was washed off in this step as expected due to the intense binding of virus to resins.

vp39 and gp64 were not detected in the fraction obtained from the washing step by western blotting (data not shown), indicating that the virus was not washed off in the step, which complied well with expectations. SDS-PAGE and protein assay (Table 6) were employed to determine the purity. Although there was an amount of protein in the fraction obtained from the washing step, none of virus envelope protein was observed. Therefore, those bands suggested that impurities were washed off. For the results of protein assay, concentration of protein was deduced to 0.63 mg/mL and 0.34 mg/mL from 4.42 mg/mL which was originally loaded. By quantitative method, it was proved that impurities were removed after virus buffer being passed through the column and washed.

TABLE 6
Determination of protein concentrations of the fractions obtained from
each of steps in affinity chromatography by using protein assay

| Fractions | V | L | FT | W1 | W2 | E1 | E2 |
|---|---|---|---|---|---|---|---|
| Protein | 8.30 | 4.42 | 3.34 | 0.63 | 0.34 | 0.0257 | 0.0035 |
| (mg/mL) | | | | | | | |

Note:
V (virus): virus in 5% FBS; L (Load): virus buffer obtained by ultrafiltration; FT: flow through; W1: wash 1 (binding buffer); W2: wash 2 (20 mM mannoside); E1: elution 1; E2: elution 2.

After washing, the elution steps were performed by using 0.6 M and 1.0 M mannoside elution solutions. As shown in Table 5, the total virus titer of virus buffer loaded into the column was 3.78×10⁸ TU. After elution with 0.6 M mannoside elution solution, the total virus titer of the eluent was 5.82×10⁷ TU and the recovery yield was 15.39%. After elution with 1 M mannoside elution solution, total virus titer of the eluent was 2.25×10⁷ TU and the recovery yield was 5.94%. Therefore, the total recovery yield from all elution steps was 21.32%.

The results of SDS-PAGE for purity analysis indicated that a few bands were present in the sample prior to being loaded into column (Load) (data not shown). After being purified by chromatography, only a part of bands were present in the eluent, indicating that lots of impurities had been removed. Furthermore, the amount of proteins before and after purification was evaluated by protein assay (Table 6). The amount of protein in the fraction originally loaded to the column (Load) was 4.42 mg/mL. The amount of proteins in the fraction obtained from the elution step with 0.6 M mannoside was 0.025 mg/mL. The amount of proteins in the fraction obtained from the elution step with 1.0 M mannoside was 0.0035 mg/mL, demonstrating that the amount of proteins obviously decreased.

To prove that the fractions obtained from the elution steps contained desired virus, the existence of virus envelope protein in the fractions obtained from the elution steps was examined by western blotting, in the fractions obtained from the elution steps (i.e. E1 and E2) gp64 and vp39 were present. This demonstrated that virus was eluted from the resins, and the amount of proteins in E1 (the fraction obtained by elution with 0.6 M mannoside elution solution) was larger than those in E2 (the fraction obtained by elution with 1.0 M mannoside elution solution), comparable to the results of recovery yield. Accordingly, the decrease of the amount of undesired proteins demonstrated that the purity of the virus was significantly increased. It was shown that Con A affinity chromatography was indeed capable of retaining a high purity of baculovirus such that the recovery yield was also greatly improved to 21% from 10% as previously reported (Hu, Y-C et al., (2003a), Enzyme Microb. Technol., 33:445-452).

Besides, the purified virus was examined by TEM analysis in the present example to discover if the purified virus retained its integrity. The figure of virus purified by Con A affinity chromatography was able to be distinctly recognized (data not shown), hence the virus retained its integrity after purification. Moreover, the results showed that intact virus could be observed in the images obtained from TEM with various magnifications.

To summarize, the purification in combination of tangential flow filtration and Con A affinity chromatography could both enhance the purity or the virus recovery yield and the virus retained its integrity after purification.

Comparing to conventional methods, the overall recovery yield in the methods of the present invention was up to 16%, which was calculated as the total virus titers of the fractions obtained from all elution steps divided by the total virus titer of the fraction originally loaded. This was a great improvement in the recovery yield, comparing to the recovery yield of purification procedures with ultracentrifugation or IMAC being less than 10%. Furthermore, the methods of the present invention were more convenient and feasible to utilize in large-scale purification. Therefore, the method for purifying baculovirus in the present invention was indeed a novel method for purifying baculovirus with enhanced efficiency.

All patents, patent applications, and literature cited in the specification were incorporated by reference in their entirety. In the case of any inconsistencies, the present disclosure, including any definitions therein will prevail.

Even though numerous characteristics and advantages of the present invention have been set forth in the foregoing description, together with details of the structure and features of the invention, the disclosure is illustrative only. Changes may be made in the details within the principles of the invention to the full extent indicated by the broad general meaning of the terms in which the appended claims are expressed.

What is claimed is:

1. A method for purifying baculovirus, comprising:
providing a baculovirus mixture containing a baculovirus and a liquid portion;
replacing the liquid portion with a binding buffer by an ultrafiltration system to form a virus buffer; and
purifying the baculovirus from the virus buffer with glycoprotein specific affinity chromatography.

2. The method as claimed in claim 1, wherein the ultrafiltration system is selected from a group consisting of stirred cell filtration and tangential flow filtration.

3. The method as claimed in claim 1, wherein the ultrafiltration system is tangential flow filtration.

4. The method as claimed in claim 1, wherein the baculovirus is selected from the group consisting of Autographa californica nucleopolyhedrovirus (AcMNPV), Bombyx mori nucleopolyhedrovirus (BmNPV), Buzura suppressaria nuclear polyhedrosis virus, Cryptophlebia leucotreta granulosis virus, Lymantria dispar baculovirus, Mamestra brassicae nuclear polyhedrosis virus, Orgyia pseudotsugata mononuclear polyhedrosis virus, Penaeus monodon-type baculovirus, Plodia interpunctella granulosis virus and Trichoplusia ni mononuclear polyhedrosis virus and modified viruses thereof.

5. The method as claimed in claim 1, wherein the baculovirus is Autographa californica nucleopolyhedrovirus (AcMNPV) and modified viruses thereof.

6. The method as claimed in claim 1, wherein the baculovirus is vesicular stomatitis virus G protein-modified Autographa californica nucleopolyhedrovirus (VSVG-AcMNPV).

7. The method as claimed in claim 2, wherein the ultrafiltration system is performed by a filter membrane with nominal molecular weight limits ranging from 100 to 500 kDa.

8. The method as claimed in claim 2, wherein the ultrafiltration system is performed by a filter membrane with nominal molecular weight limit of 300 kDa.

9. The method as claimed in claim 3, wherein the tangential flow filtration is performed under pressure ranging from 2 to 10 psi.

10. The method as claimed in claim 3, wherein replacement of the liquid portion of the baculovirus mixture is performed at a temperature ranging from 15° C. to 35° C.

11. The method as claimed in claim 3, wherein replacement of the liquid portion of the baculovirus mixture is performed at a temperature ranging from 20° C. to 30° C.

12. The method as claimed in claims 1 to 3, wherein the binding buffer is a solution containing 20 mM Tris, 0.5 M NaCl, 1 mM CaCl₂ and 1 mM MnCl₂ at pH 7.4.

13. The method as claimed in claims 1 to 3, wherein the affinity chromatography is performed with lectin-conjugated resins.

14. The method as claimed in claim 13, wherein the lectin is selected from the group consisting of Concanavalin A (Con A), Lentil lectin and wheat germ agglutinin (WGA).

15. The method as claimed in claim 13, wherein the glycoprotein specific affinity chromatography is performed at a temperature ranging from 15° C. to 35° C.

16. The method as claimed in claim 13, wherein the glycoprotein specific affinity chromatography is performed at a temperature ranging from 20° C. to 30° C.

17. The method as claimed in claim 13, wherein the glycoprotein specific affinity chromatography is performed with an elution solution containing saccharides.

18. The method as claimed in claim 17, wherein the saccharides of the elution solution are selected from the group consisting of mannoside or derivatives thereof.

19. The method as claimed in claim 17, wherein the elution solution comprises α-methyl-D-mannoside at a concentration ranging from 0.5 M to 1.2 M.

20. The method as claimed in claim 19, wherein the elution solution further contains 0.5 M NaCl.
